(19) United States
(12) Patent Application Publication
(10) Pub. No.: US 2026/0060882 A1
(43) Pub. Date: Mar. 5, 2026

(54) HANDHELD MASSAGE DEVICE

(71) Applicant: Shenzhen Light Tree Medical Technology Co., Ltd., Shenzhen (CN)

(72) Inventors: Alain Dijkstra, Amstelveen (NL); Kim Laurens Markwat, Den Haag (NL); Michael Kasbergen, Schoonhoven (NL); Wang Jingjue, Shenzhen (CN); Wang Dezhi, Shenzhen (CN)

(73) Assignee: Shenzhen Light Tree Medical Technology Co., Ltd., Shenzhen (CN)

(21) Appl. No.: 19/319,795

(22) Filed: Sep. 5, 2025

(30) Foreign Application Priority Data
Sep. 5, 2024 (CN) 2024112474017
Sep. 5, 2024 (CN) 2024221846495

(51) Int. Cl. A61H 15/02; A61H 15/00; A61N 1/32; A61N 2/00; A61N 5/06

(52) U.S. Cl. CPC A61H 15/02; A61H 15/0092; A61N 1/322; A61N 1/328; A61N 2/002; A61N 5/0616; A61H 2015/005; A61H 2015/0071; A61H 2201/0207; A61H 2201/0214; A61H 2201/0228; A61H 2201/0285; A61H 2201/10; A61H 2201/1671; A61H 2201/1695; A61N 2005/0644; A61N 2005/0651; A61N 2005/0659; A61N 2005/0663

(57) ABSTRACT

The invention relates to the technical field of a handheld massage instrument, providing a handheld device comprising a handle and a massage mechanism. The massage mechanism comprises an insulating mounting piece, a conductive connector, a conductive ball, and a conductive massage piece. The conductive massage piece is rotatably supported by the insulating mounting piece and configured to contact a user's skin. The conductive connector is electrically connected to the conductive massage piece and is configured to receive power from a power source accommodated within the handle. The conductive ball is disposed between the conductive connector and the conductive massage piece, and is configured to maintain rolling contact with the conductive connector and the conductive massage piece, thereby enabling smooth rotation while ensuring stable electrical conduction.

### TECHNICAL FIELD

The present invention relates to the technical field of therapeutic and cosmetic treatment devices, and more particularly to a handheld massage and electrotherapy device configured for application to a user's skin. Specifically, the invention provides a handheld massage device configured for the therapeutic or cosmetic treatment, incorporating features such as massage, electrotherapy, mechanical stimulation, etc.

### BACKGROUND

Massage devices are widely used in the field of physiotherapy, rehabilitation, and personal healthcare to relieve muscle fatigue, improve blood circulation, and provide therapeutic stimulation to the human body. In particular, handheld massage devices configured to deliver both mechanical massage and electrical stimulation (electrotherapy) have gained attention due to their potential to combine physical and bioelectrical benefits in a single device.

The handheld massage devices typically include a handle portion and one or more massage heads electrically coupled to a power source. The massage heads may be made of conductive materials and designed to contact the skin of a user while transmitting a therapeutic current. Although these conventional devices are functional, there remain several shortcomings.

In conventional devices, the massage head is mounted directly to a shaft or connector without adequate limiting structures such as grooves, stop portions, or positioning features. As a result, the massage head may loosen, wobble, or shift axially or circumferentially during use, thereby reducing stability, user comfort, and durability of the product.

The existing devices often rely on direct sliding contact between the conductive massage head and the conductive connector for electrical transmission. Such arrangements are prone to frictional wear, uneven conduction, sparking, or localized heating, which can degrade performance over time and compromise safety. The lack of a stable and uniform current path further reduces the effectiveness of electrotherapy.

Furthermore, the rotational movement of massage heads in prior devices is hindered by surface-to-surface friction between contacting components. This results in jerky or uneven operation, high resistance to rotation, and premature wear of conductive elements. Some attempts to improve smoothness have introduced complex bearing or gear structures, which increase manufacturing cost, assembly difficulty, and device size, thereby reducing suitability for handheld operation.

Additionally, many prior designs expose conductive elements or provide insufficient insulation between electrical parts and the external environment. Such shortcomings can create risks of current leakage, moisture ingress, or accidental electric shock, raising safety concerns for end users, especially in home-use environments.

Furthermore, conventional massage heads are often rigid and unable to adapt to varying body contours. The lack of flexible or deformable structures reduces the effectiveness of massage on curved or sensitive areas of the body, thereby limiting therapeutic benefits.

Accordingly, there remains a need for a handheld massage device that ensures stable assembly of the massage mechanism, provides smooth rotation with reliable electrical conduction, incorporates adequate insulation for safety, and adapts comfortably to the contours of the human body, while maintaining a compact and ergonomic structure.

### OBJECTS OF THE INVENTION

It is an object of the present invention to provide a handheld massage device that overcomes the disadvantages of existing devices, wherein the massage mechanism does not rotate smoothly and cannot reliably transmit microcurrents.

It is another object of the invention to provide a massage device with a structurally stable massage mechanism that ensures smooth, continuous, and durable rolling rotation during massage.

It is a further object of the invention to reduce friction and wear between the conductive parts, insulating components, and massage elements so as to prolong the service life of the device.

It is still another object of the invention to provide an improved conduction pathway that enables stable and reliable transmission of microcurrents from the handle to the massage mechanism for enhanced therapeutic efficacy.

It is an additional object of the invention to simplify assembly and disassembly of the massage mechanism by incorporating detachable mounting parts, guiding grooves, and limiting structures that improve manufacturability and maintenance convenience.

It is another object of the invention to provide a lightweight, portable, and battery-powered handheld massage device capable of delivering at least a physical massage or electrotherapy.

### SUMMARY OF THE INVENTION

According to a first aspect of the present invention, a handheld massage device is provided. The handheld massage device comprising: a handle including a gripping portion and at least two connecting branches extending from the gripping portion arranged at an angle with respect to one another; a first massage mechanism disposed on a first connecting branch and electrically connected to a positive terminal of a power supply; and a second massage mechanism disposed on a second connecting branch and electrically connected to a negative terminal of the power supply, wherein the first massage mechanism and the second massage mechanism are configured to contact the human body simultaneously to provide at least one physical massage or electrotherapy.

In one embodiment of the invention, at least two connecting branches are symmetrically arranged relative to a central axis of the gripping portion.

According to a second aspect of the present invention, a handheld massage device is provided. The handheld massage device comprising: a handle including a gripping portion and at least two connecting branch extending from the gripping portion, each of the at least two connecting branch is mounting with a massage mechanism; wherein each massage mechanism comprises: an insulating mounting piece; a conductive massage piece rotatably supported by the insulating mounting piece and configured to contact a user's skin; a conductive connector electrically connected to the conductive massage piece, configured to receive power from a power source accommodated within the handle; and a conductive ball disposed between the conductive connector and the conductive massage piece, the conductive ball being in rolling contact with both the conductive connector and the conductive massage piece to facilitate smooth rotation and stable electrical conduction.

In one embodiment of the invention, the handheld massage device further comprising an insulating housing disposed between the conductive massage piece and the insulating mounting piece.

In one embodiment of the invention, the conductive massage piece is rotatably fitted onto the insulating mounting piece by a sleeve-and-groove arrangement, the groove accommodating the conductive ball for smooth rotation.

In one embodiment of the invention, the conductive massage piece comprises a conductive sleeve, at least one elastic arm, and one or more massage protrusions, wherein the at least one elastic arm connects the conductive sleeve to the one or more massage protrusions to provide flexible deformation.

In one embodiment of the invention, the conductive sleeve has at least one axial deformation gap forming a non-closed ring to allow elastic deformation.

In one embodiment of the invention, the insulating housing is fixedly sleeved around the conductive sleeve, the housing having a through hole through which the massage protrusion extends.

In one embodiment of the invention, the insulating mounting piece comprises at least one groove and a stop portion configured to limit axial or circumferential movement of the conductive massage piece.

In one embodiment of the invention, the groove and the stop portions maintain stable rotational alignment of the conductive massage piece and conductive ball.

In one embodiment of the invention, the conductive connector is sleeved onto the insulating mounting piece and electrically connected to the power supply.

In one embodiment of the invention, the conductive connector is in rolling contact with the conductive ball and the conductive massage piece throughout rotation.

In one embodiment of the invention, the plurality of conductive balls is disposed circumferentially between the conductive connector and the conductive massage piece to provide multi-point electrical conduction and rotational stability.

In one embodiment of the invention, the conductive connector and insulating mounting piece comprise cooperating limiting grooves or connecting holes to secure the conductive connector relative to the insulating mounting piece.

In one embodiment of the invention, the conductive massage piece comprises a second groove on an inner side, the conductive ball being partially confined within the second groove.

In one embodiment of the invention, the conductive massage piece rotates relative to the handle while the conductive ball maintains rolling contact to reduce friction and wear.

In one embodiment of the invention, the handheld massage device further comprising one or more stimulation elements in each of the massage mechanisms to provide one or more therapies to the user.

In one embodiment of the invention, the stimulation element is selected from a group consisting of, but is not limited to a phototherapy, micro-current, magneto therapy, cooling, heating, vibration, electrical pulses, or other forms of therapeutic output.

According to a third aspect of the present invention, a method of massaging and applying electrotherapy to a user's skin is provided. The method comprising: providing a handheld massage device having a handle with a power source, at least one conductive massage piece, and an insulating mounting piece supporting the conductive massage piece; transmitting electrical current from the power source through a conductive connector and a conductive ball to the conductive massage piece; and contacting the conductive massage piece with the user's skin to provide at least one of the massage or electrotherapy.

In one embodiment of the invention, the conductive ball is housed in a groove of the insulating mounting piece to provide positional stability.

In the context of the specification, the term “comprising” (and grammatical variations thereof) is intended to be open-ended, meaning that the listed elements or steps are not exhaustive and do not exclude the presence of additional, unrecited elements or steps.

In the context of the specification, the terms “a,” “an,” and “the” are intended to cover both the singular and plural forms, unless otherwise indicated by context.

In the context of the specification, the term “or” is used inclusively, unless stated otherwise, meaning “and/or.”

In the context of the specification, the terms such as “axial,” “circumferential,” “radial,” “inner,” “outer,” “upper,” “lower,” “front,” “rear,” and the like are used for convenience to describe relative orientation and do not impose any absolute limitations on device orientation during use.

In the context of the specification, the term “groove” shall encompass any recessed channel, slot, indentation, or track formed on a component, regardless of cross-sectional shape.

In the context of the specification, the term “stop portion” includes any protrusion, shoulder, ledge, or surface configured to limit movement of an adjacent component.

In the context of the specification, the term “handle” shall encompass any housing, shank, body, casing, or support structure suitable for gripping by a user and for accommodating electrical or mechanical components.

In the context of the specification, the term “insulating mounting piece” refers to any structural element made from electrically non-conductive material that supports, aligns, or positions conductive elements and massage mechanisms.

In the context of the specification, the term “conductive connector” refers to any electrically conductive element configured to couple the massage mechanism to a power source, including but not limited to sleeves, terminals, rings, clips, or pins.

In the context of the specification, the term “conductive ball” shall encompass any rolling element, bead, or spherical conductor disposed to provide both electrical contact and rotational support between adjacent parts. The conductive ball may be a single element or a plurality of elements arranged circumferentially.

In the context of the specification, the term “conductive massage piece” refers to any element intended to contact a user's skin to provide mechanical massage and/or electrical stimulation. This may include, without limitation, sleeves, shells, rollers, protrusions, or combinations thereof.

The term “insulating housing” shall include any structure enclosing or partially enclosing conductive components to provide insulation, protection against contamination, or mechanical stability.

In the context of the specification, the term “massage protrusion” refers broadly to any raised element, nub, ridge, or projection on the massage piece adapted to contact the user's skin.

In the context of the specification, the term “power source” includes but is not limited to batteries, rechargeable cells, capacitors, or external power supplies.

In the context of the specification, the term “electrotherapy,” “electrical stimulation,” or “microcurrent therapy” refers to the application of therapeutic current to the human body, regardless of the precise frequency, waveform, or current intensity.

In the context of the specification, the term “electrically connected” shall include direct and indirect electrical coupling, including via intermediate conductive elements, rolling contacts, or flexible connectors.

In the context of the specification, the term “electrotherapy” may refer to different types of electrotherapy, including but not limited to galvanic, electrotherapy, iontophoresis, microcurrent, and EMS (Electrical Muscle Stimulation), TENS (Transcutaneous Electrical Nerve Stimulation).

In the context of the specification, the term “stimulation element” refers to may include, but is not limited to, phototherapy, micro-current, magneto therapy, cooling, heating, vibration, electrical pulses, or other forms of therapeutic output.

### BRIEF DESCRIPTION OF THE ACCOMPANYING DRAWINGS

In order to more clearly illustrate the technical solutions in the embodiments of the present invention, the drawings that need to be used in the embodiments or prior art descriptions will be briefly introduced below, and it is obvious that the drawings in the following descriptions are only some embodiments of the present invention, and for those skilled in the art, other drawings can also be obtained according to these drawings without paying creative labor.

FIG. 1 is a schematic structural diagram of the handheld massage device according to one embodiment of the present invention.

FIG. 2 is a schematic structural diagram of the massage mechanism of the handheld massage device.

FIG. 3 is a cross-sectional view of the massage mechanism of FIG. 2 taken along line A-A.

FIG. 4 is a partially enlarged view of portion A in FIG. 3.

FIG. 5 is another view of the massage mechanism of FIG. 2.

FIG. 6 is an exploded view of the massage mechanism of the handheld massage device.

FIG. 7 is a schematic structural view of the middle cage of the massage mechanism shown in FIG. 6.

FIG. 8 is a schematic structural view of the conductive massage piece of the massage mechanism shown in FIG. 6.

FIG. 9 is a schematic structural view of the handle of the handheld massage device.

FIG. 10 is a cross-sectional view of the handle shown in FIG. 9.

### DETAILED DESCRIPTION

Embodiments of the present invention disclosure will be described more fully hereinafter with reference to the accompanying drawings in which like numerals represent like elements throughout the figures, and in which example embodiments are shown.

The detailed description and the accompanying drawings illustrate the specific exemplary embodiments by which the disclosure is practiced. These embodiments are described in detail to enable those skilled in the art to practice the invention illustrated in the disclosure. It is to be understood that other embodiments are utilized, and other changes are made, without departing from the spirit or scope of the present disclosure. The following detailed description is therefore not to be taken in a limiting sense, and the scope of the present invention disclosure is defined by the appended claims. Embodiments of the claims, however, may be embodied in many different forms and should not be construed as limited to the embodiments set forth herein.

It should be noted that when an element is referred to as being “fixed to” or “disposed on” another element, it is directly or indirectly attached to the other element. When an element is referred to as being “connected to” another element, it is directly or indirectly connected to the other element.

Embodiments of the present invention disclose a handheld massage device configured to deliver both physical massage and therapeutic stimulation to a user. The device generally comprises a handle and at least two massage mechanisms mounted thereon. The handle includes a gripping portion that is ergonomically shaped to be comfortably held by a user, and at least two connecting branches extending from the gripping portion. The connecting branches may be disposed at an angle relative to one another and may be symmetrically arranged about a central axis of the handle. Each connecting branch supports a respective massage mechanism, and the two massage mechanisms may be electrically connected to opposite terminals of a power supply housed within the handle such that, when the massage mechanisms contact the human body simultaneously, a closed microcurrent circuit is formed through the body to provide electrotherapy in addition to massage.

Each massage mechanism comprises an insulating mounting piece that is fixedly mounted on the corresponding connecting branch of the handle. The insulating mounting piece may be formed of a dielectric material such as polycarbonate, ABS, polypropylene, polyamide, ceramic, or composites thereof. The insulating mounting piece provides structural support and electrical isolation for the conductive components of the massage mechanism. A conductive connector is mounted on or sleeved around the insulating mounting piece and is electrically coupled to the power supply located within the handle. The conductive connector transmits electrical current to a conductive massage piece, which is rotatably supported on the insulating mounting piece and configured to contact a user's skin.

A conductive ball is disposed between the conductive connector and the conductive massage piece. The conductive ball is maintained in rolling contact with both the conductive connector and the conductive massage piece, thereby enabling stable electrical conduction while simultaneously permitting smooth relative rotation. In some embodiments, a plurality of conductive balls is circumferentially distributed between the conductive connector and the conductive massage piece, thereby ensuring multi-point conduction and enhanced rotational stability. To further ensure positioning, the insulating mounting piece may include a circumferential groove in which a portion of the conductive ball is retained, while the conductive connector may include a limiting hole through which the conductive ball partially protrudes to engage the conductive massage piece. Stop portions or cooperating groove structures may be provided between the insulating mounting piece and the conductive connector to restrict unwanted axial movement.

The conductive massage piece itself may be formed as a conductive sleeve having one or more elastic arms connected to massage protrusions. The elastic arms allow controlled deformation, enabling the massage protrusions to flex under applied pressure and adapt to the contours of the user's skin. In some embodiments, the conductive sleeve may include an axial deformation gap or a combination of linear and arc-shaped gaps forming a non-closed ring structure, allowing the sleeve to expand or contract slightly for comfort during massage. The massage protrusions extend outward, and in certain embodiments may pass through corresponding through-holes of an insulating housing or shell that surrounds the conductive sleeve. The insulating housing serves to provide electrical insulation, protect the conductive components from external contaminants, and offer structural reinforcement.

The handle of the device houses a power supply such as a battery, which may be rechargeable, along with a circuit board that regulates the delivery of electrical current to the conductive connectors of the massage mechanisms. The two massage mechanisms may thus be driven with opposite polarities to form a closed-loop microcurrent circuit through the user's body when both massage mechanisms are simultaneously applied to the skin. In some embodiments, the handheld massage device may further be enhanced with additional stimulation elements integrated into the massage mechanisms or handle. For example, a phototherapy module comprising one or more light-emitting diodes may be disposed on the insulating housing or embedded near the massage protrusions. The phototherapy module may emit therapeutic wavelengths, such as red, blue, near-infrared, or combined spectrums, to promote skin rejuvenation, improve circulation, and reduce inflammation during massage.

In another embodiment, the device may incorporate magnetotherapy features by embedding permanent magnets or electromagnets within the conductive massage piece, the insulating mounting piece, or the handle. These magnets generate a localized magnetic field that works in conjunction with massage and electrical stimulation to promote cellular activity, relieve pain, and enhance recovery.

Further, the device may be equipped with thermal modulation components. Heating may be provided by resistive heating elements disposed within or adjacent to the conductive massage piece to deliver localized warmth, thereby relaxing muscles and enhancing blood flow. Conversely, cooling may be provided by a thermoelectric module such as a Peltier element integrated into the massage mechanism to reduce swelling, soothe inflammation, and provide cryotherapy effects.

The massage device may also include a vibration module integrated into the handle or massage mechanism. The vibration module may comprise an eccentric rotating mass motor or a linear resonant actuator to impart oscillatory motion to the massage protrusions, thereby intensifying the massage experience and facilitating deeper tissue penetration.

In yet another embodiment, the massage device may be configured to deliver pulsed electrical stimulation. The conductive connectors and massage pieces may be driven by a control circuit that generates monophasic, biphasic, or multiplexed current waveforms. The pulsed electrical stimulation may be used alone or in combination with phototherapy, vibration, or thermal modulation, enabling customized therapy programs.

The various stimulation components may be implemented individually or in combination, depending on the desired therapeutic outcome. For example, one embodiment may simultaneously deliver microcurrent electrotherapy through the conductive massage pieces, phototherapy through integrated LEDs, and vibration via a motor in the handle. Another embodiment may combine heating elements with magnetotherapy to provide simultaneous warmth and magnetic stimulation. Still another embodiment may combine cooling therapy with pulsed electrical stimulation for post-exercise recovery.

The device may be manufactured as a modular platform, enabling different stimulation modules to be included or omitted depending on the intended application or target market. This modularity allows for cost-effective manufacturing of both basic and advanced models, ensuring that the invention can be adapted for use in cosmetic skincare, pain management, muscle relaxation, or rehabilitation therapy.

In an embodiment of the present invention, a method for delivering a massage treatment or electrotherapy to a user's skin using this device is provided using the handheld massage therapy device. While using the handheld massage device is gripped by the user at the gripping portion of the handle, with the at least two connecting branches extending outwardly to position the respective massage mechanisms for skin contact. The user applies both massage mechanisms to different regions of the body simultaneously, such as opposite sides of the face, neck, shoulders, arms, or other treatment areas.

When the massage mechanisms are brought into contact with the skin, the conductive massage pieces supported by the insulating mounting pieces engage the user's skin surface. The conductive balls, maintained in rolling contact between the conductive connectors and the conductive massage pieces, allow the massage pieces to rotate smoothly while maintaining reliable conduction of electrical current. As a result, electrical energy from the power source housed within the handle is transmitted through the conductive connector and conductive ball into the conductive massage piece, and then into the user's skin. Because the first massage mechanism and the second massage mechanism are connected to opposite terminals of the power supply, a closed electrical loop is formed through the user's body, thereby delivering microcurrent or other therapeutic electrical stimulation while simultaneously performing physical massage.

During operation, the user may move the handheld massage device along the contours of the skin, allowing the massage protrusions on the conductive massage pieces to apply localized pressure and rolling massage. The elastic arms of the massage protrusions provide flexible deformation, enabling the protrusions to adapt to varying surface contours of the body, thus enhancing comfort and coverage. The insulating shell, when provided, ensures that only the intended massage protrusions contact the skin, maintaining user safety and controlled application of current.

In certain embodiments, the method further comprises delivering additional stimulation modalities in combination with the massage and microcurrent therapy. For example, the device may emit light from phototherapy modules disposed near the massage protrusions, thereby irradiating the skin with red, blue, or near-infrared wavelengths during massage to promote collagen synthesis, reduce acne, or enhance circulation.

In another embodiment, the method comprises applying localized magnetic fields by virtue of magnetotherapy elements embedded in the massage mechanisms or handle. During massage, these magnetic fields interact with the tissues beneath the skin, providing an additional therapeutic effect such as reduction of muscle fatigue or pain relief.

In further embodiments, the method comprises delivering controlled heating or cooling. Heating elements integrated into the conductive massage pieces may be activated to warm the skin and underlying muscles, enhancing relaxation and blood flow, while cooling elements such as Peltier modules may be activated to lower tissue temperature, reduce inflammation, or promote recovery after exercise.

In yet another embodiment, the method comprises imparting vibration to the massage mechanisms through activation of a vibration motor housed in the handle. The vibration is transmitted to the massage protrusions in contact with the skin, thereby augmenting the physical massage action and increasing stimulation of deeper tissues.

In some variations, the method further comprises delivering pulsed or patterned electrical stimulation, wherein the control circuit housed within the handle generates specific waveforms of current, such as monophasic, biphasic, or multiplexed pulses. These controlled pulses may be applied through the conductive massage pieces to achieve specific therapeutic effects such as pain relief, neuromuscular stimulation, or improved tissue healing.

The method for providing massage therapy or electrotherapy comprises: (1) positioning the massage mechanisms against the user's skin; (2) activating the device to transmit current through the conductive ball and conductive massage pieces into the skin; (3) moving the device along the treatment area to provide physical massage; (4) selectively activating additional stimulation modules, such as phototherapy, magnetotherapy, heating, cooling, vibration, or pulsed current; and (5) continuing treatment for a predetermined duration according to therapeutic requirements.

Accordingly, the method of using the handheld massage device allows simultaneous application of physical massage and electrotherapy while further enabling optional combinations of phototherapy, magnetotherapy, thermal modulation, vibration, and pulsed current stimulation. The method provides a versatile treatment protocol that can be tailored to cosmetic, therapeutic, rehabilitative, or relaxation applications.

Several embodiments of the present invention will now be described in detail with references to FIGS.

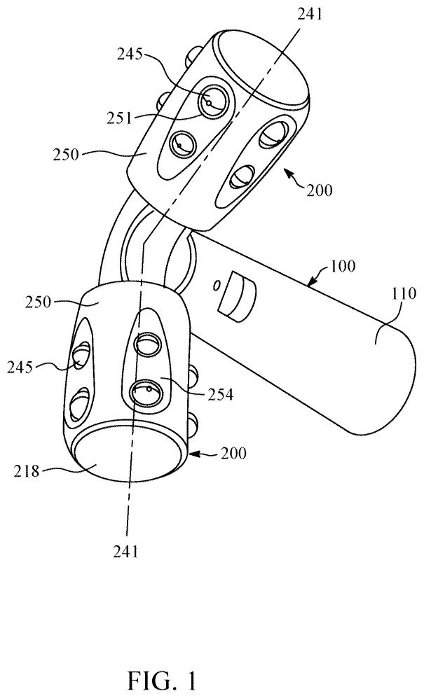

Referring to FIGS. 1, 2, and 3, the present application provides a handheld massage device. The handheld massage device comprises a handle 100 and a massage mechanism 200. The massage mechanism 200 comprises an insulating mounting piece 210, a conductive connector 220, a conductive ball 230, and a conductive massage piece 240. The insulating mounting piece 210 is fixedly mounted on the handle 100. The conductive connector 220 is installed on the insulating mounting piece 210. The conductive connector 220 is configured to receive power from a power source accommodated within the handle 100. The conductive massage piece 240 is rotatably supported by the insulating mounting piece 210 and configured to contact a user's skin. The conductive ball 230 is disposed between the conductive connector 220 and the conductive massage piece 240. The conductive ball 230 is in rolling contact with both the conductive connector 220 and the conductive massage piece 240 to facilitate smooth rotation and stable electrical conduction.

In this embodiment, the insulating mounting piece 210 is fixedly mounted on the handle 100 by a mounting method selected from the group consisting of interference fit, threaded engagement, adhesive bonding, ultrasonic welding, snap-fit connection, or a combination of adhesive bonding and mechanical fastening.

In this embodiment, the current is rotated around the insulating mounting piece 210 through the conductive connector 220, the conductive ball 230, and the conductive massage piece 240, and is in rolling contact with the human body. A conductive ball 230 is provided between the conductive massage piece 240 and the insulated mounting piece 210. The rotational resistance and wear of the conductive massage piece 240 are greatly reduced, so that the massage mechanism 200 is smoother when rotating. At the same time, the conductive massage piece 240 that rotates smoothly is conducive to the uniform conduction of microcurrent, enhances the uniformity and depth of massage, and improves massage comfort.

In this embodiment, the conductive ball 230 is mounted on the insulating mounting piece 210 at a rolling limit, or it is installed in the conductive connector 220. The conductive ball 230 can also be installed in the insulating mounting piece 210 and the conductive connector 220, and there is no restriction herein.

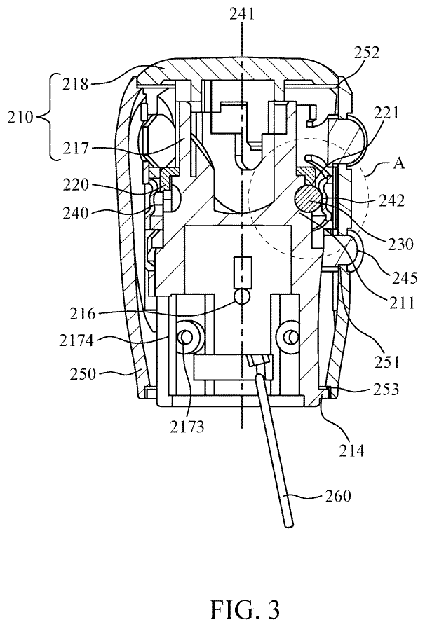

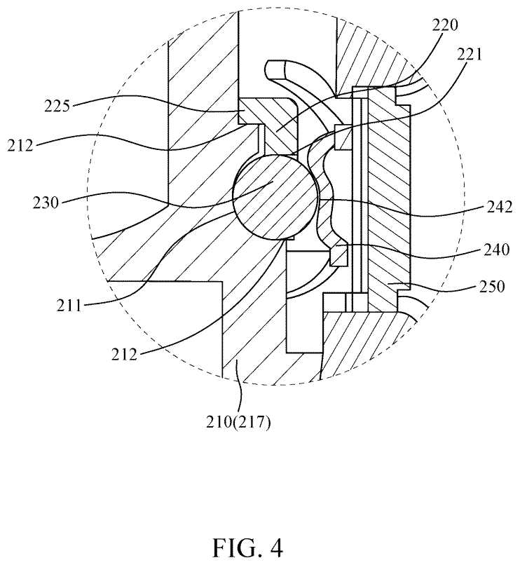

In some embodiments, and with reference to FIGS. 3 and 4, the insulating mounting piece 210 is provided with a first groove 211. The first groove 211 is configured to accommodate the circumferential extension of the conductive ball 230 within the insulating mounting piece 210, thereby achieving axial support and positional restriction of the conductive ball 230. This arrangement ensures the axial stability and operational reliability of both the conductive ball 230 and the conductive massage piece 240 during use, while also eliminating the need for rigid connection operations and thereby simplifying the assembly process.

In addition, a portion of the conductive ball 230 is exposed through the first groove 211. On the one hand, the first groove 211 is not required to enclose the entire conductive ball 230, which facilitates a reduced overall size of the first groove 211. On the other hand, the exposed portion of the conductive ball 230 enables easier electrical connection with the conductive connector 220 and the conductive massage piece 240. During rolling contact, the conductive connector 220 and the conductive massage piece 240 do not need to extend into the first groove 211 to maintain electrical conduction with the conductive ball 230.

Specifically, the bottom surface of the first groove 211 is formed as a spherical groove wall, wherein the diameter of the sphere defining the spherical groove wall is greater than the diameter of the conductive ball 230.

The conductive ball 230 abuts against the spherical groove wall, such that the contact is mainly concentrated on the curved surface of the conductive ball 230 rather than over its entire surface. This configuration reduces the contact area between the conductive ball 230 and the groove wall during rolling, thereby decreasing wear and rolling resistance.

The diameter of the sphere defining the spherical groove wall is 2 to 5 times the diameter of the conductive ball 230. Within this range, the conductive ball 230 is properly positioned in the first groove 211 while minimizing oscillation relative to the rotational axis 241 of the conductive massage piece 240.

If the sphere diameter is less than twice the diameter of the conductive ball 230, the clearance between the opening of the first groove 211 and the conductive ball 230 becomes too small. This causes the conductive ball 230 to grind against the groove edge during rolling, increasing wear, while also excessively restricting the swing flexibility of the conductive ball 230 relative to the rotational axis 241, thereby impairing rolling smoothness.

Conversely, if the sphere diameter is greater than five times the diameter of the conductive ball 230, the support and positional restriction provided by the first groove 211 becomes insufficient, reducing the stability of the conductive ball 230 during rolling and potentially generating noise and vibration.

The radius of the conductive ball 230 is greater than the groove depth of the first groove 211, thereby preventing the conductive ball 230 from being excessively embedded within the groove. This avoids unnecessary friction and wear while improving rolling flexibility. For example, the groove depth of the first groove 211 is 1.2 to 2 times smaller than the radius of the conductive ball 230, ensuring that the conductive ball 230 maintains an appropriate clearance and degree of freedom for smooth and stable rolling.

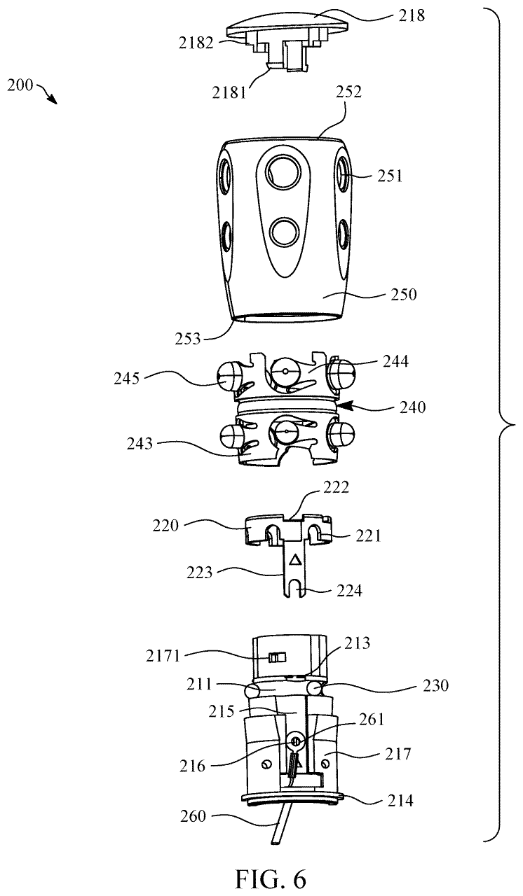

In some embodiments, with reference to FIGS. 4 and 6, the conductive connector 220 covers the opening of the first groove 211 and is provided with a limiting hole 221. The conductive ball 230 is partially exposed through the limiting hole 221 to the exterior of the conductive connector 220. The limiting hole 221 provides circumferential restriction for the conductive ball 230, thereby stabilizing its position within the insulating mounting piece 210 and ensuring stable rolling contact with the conductive massage piece 240. This enhances both conductive stability and rotational smoothness of the conductive massage piece 240.

Specifically, the limiting hole 221 is dimensioned to form a clearance fit with the conductive ball 230. This configuration allows smooth rolling of the conductive ball 230, while also enabling the conductive massage piece 240 to drive the conductive ball 230 to rotate circumferentially around its rotation axis 241. As a result, the conductive ball 230 maintains stable rolling contact with the inner wall of the limiting hole 221, thereby ensuring stable conductivity. The diameter of the limiting hole 221 is 1.01 to 1.5 times the diameter of the conductive ball 230. This ratio ensures sufficient rolling flexibility of the conductive ball 230 while also providing circumferential restriction to stabilize its position.

In other embodiments, the diameter of the limiting hole 221 is less than or equal to the diameter of the conductive ball 230, such that the conductive ball 230 only partially passes through the limiting hole 221. In this manner, both circumferential and radial restrictions are enhanced, and the conductive ball 230 is simultaneously limited at the position of the limiting hole 221 during rolling.

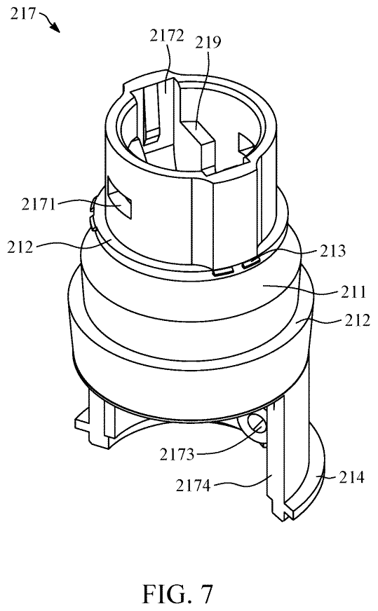

Specifically, with reference to FIGS. 6 and 7, the limiting hole 221 is open on one side adjacent to the handle 100, such that the conductive ball 230 is inserted through the open side of the limiting hole 221, thereby simplifying assembly operations.

It should be noted that the number of limiting holes 221 is greater than or equal to the number of conductive balls 230. In other words, each limiting hole 221 receives a conductive ball 230, or only a portion of the limiting holes 221 is provided with conductive balls 230, while others remain empty, without limitation. In this way, the user flexibly adjusts the number of conductive balls 230 according to the desired rotational performance of the conductive massage piece 240.

For example, when enhanced electrical conductivity of the conductive massage piece 240 is desired, the number of conductive balls 230 is increased, such that each limiting hole 221 contains a conductive ball 230. Conversely, when the conductive massage piece 240 is intended to provide a gentler massage to the human body, the number of conductive balls 230 is reduced by leaving some limiting holes 221 without conductive balls 230.

In some embodiments, with reference to FIGS. 4, 6, and 7, the insulating mounting piece 210 is provided with a first stop portion 212. The conductive connector 220, when sleeved onto the outer side of the insulating mounting piece 210, is radially stabilized relative to the insulating mounting piece 210. The conductive connector 220 abuts against the first stop portion 212, thereby restricting axial movement of the conductive connector 220 toward the handle 100. In this way, the first stop portion 212 provides axial support and positional stability of the conductive connector 220 on the insulating mounting piece 210, ensuring reliable structural and conductive performance.

Specifically, with reference to FIG. 4, the insulating mounting piece 210 is provided with a first stop portion 212 positioned on the upper and lower sides of the conductive ball 230, i.e., on opposite sides of the first groove 211. The lower end surface of the conductive connector 220 abuts against the upper end of the lower first stop portion 212, while the conductive connector 220 is further provided with a flange 225 that abuts against the upper first stop portion 212. This configuration enhances the axial supporting force and positional stability of the conductive connector 220 relative to the insulating mounting piece 210.

The end of the flange 225 is in contact with the outer wall of the insulating mounting piece 210, thereby further fixing the conductive connector 220 in the radial direction relative to the insulating mounting piece 210.

Specifically, each first stop portion 212 is formed as a step surface, wherein the normal direction of the step surface is aligned with the rotational axis 241 of the conductive massage piece 240. The conductive connector 220 is restricted by the stop step surface and prevented from further axial displacement along the insulating mounting piece 210 toward the handle 100, thereby ensuring stable positioning.

In some embodiments, with reference to FIGS. 6 and 7, one of the insulating mounting piece 210 and the conductive connector 220 is provided with a first limiting groove 222, while the other is provided with a first limiting portion 213. The first limiting portion 213 engages with the first limiting groove 222, thereby restricting axial rotation of the conductive connector 220 relative to the insulating mounting piece 210. This configuration enhances the circumferential stability of the conductive connector 220 and prevents the conductive connector 220 from rotating around the rotational axis 241 of the conductive massage piece 240 together with the conductive member.

In this way, rotational load and rolling resistance are reduced, conductivity between components is improved, and the positional stability of the conductive connector 220 is maintained. Consequently, a stable electrical connection with the power supply is ensured, thereby improving the reliability of current transmission.

Specifically, with reference to FIGS. 6 and 7, the insulating mounting piece 210 is provided with a first limiting portion 213, and the conductive connector 220 is provided with a first limiting groove 222. The flange 225 of the conductive connector 220 defines the first limiting groove 222, while the upper first stop portion 212 of the insulating mounting piece 210 is provided with the first limiting portion 213. In some embodiments, the first limiting portion 213 protrudes from the periphery of the insulating mounting piece 210, and the bottom surface of the first limiting portion 213 abuts against the bottom of the first limiting groove 222, thereby restricting axial displacement of the conductive connector 220 in a direction away from the handle 100 along the axial direction of the insulating mounting piece 210.

Furthermore, when the first stop portion 212 restricts the conductive connector 220 from further axial movement along the insulating mounting piece 210 toward the handle 100, the conductive connector 220 is fixed in position axially. At the same time, by being sleeved over the insulating mounting piece 210, radial fixation is achieved. In addition, engagement between the first limiting portion 213 and the first limiting groove 222 provides circumferential fixation, thereby further enhancing the positional stability of the conductive connector 220.

In other embodiments, the arrangement is reversed, i.e., the conductive connector 220 is provided with the first limiting portion 213 and the insulating mounting piece 210 is provided with the first limiting groove 222, without limitation.

Specifically, the first limiting portion 213 is formed as a limiting block or a limiting rib, and no particular limitation is imposed herein. In addition, a plurality of first limiting grooves 222 is provided, distributed circumferentially at intervals along the insulating mounting piece 210, to improve the uniformity and strength of circumferential positioning.

In some embodiments, with reference to FIGS. 6 and 7, the insulating mounting piece 210 is provided with a first connecting hole 216, and the conductive connector 220 is provided with a second connecting hole 224. The first connecting hole 216 and the second connecting hole 224 are configured to receive the same fastener for threaded connection, thereby permanently mounting the conductive connector 220 onto the insulating mounting piece 210.

In some embodiments, with reference to FIGS. 6 and 7, the insulating mounting piece 210 further includes a second limiting groove 215, disposed on the side of the conductive ball 230 adjacent to the handle 100. The conductive connector 220 is provided with a connecting strip 223, which is embedded within the second limiting groove 215. The connecting strip 223 abuts against the circumferential groove wall of the second limiting groove 215, thereby reinforcing the circumferential positional stability of the conductive connector 220 relative to the insulating mounting piece 210.

The second limiting groove 215 and the first limiting portion 213 are respectively arranged on opposite axial sides of the first groove 211, such that the conductive connector 220 is circumferentially restricted on both the upper and lower sides of the first groove 211.

The groove bottom of the second limiting groove 215 is provided with the first connecting hole 216, while the connecting strip 223 is provided with the second connecting hole 224. A fastener passes through both the first connecting hole 216 and the second connecting hole 224 to fixedly secure the connecting strip 223 within the second limiting groove 215. Furthermore, the connecting strip 223 is electrically connected to a power supply via a conductive transmitting piece 260. The conductive transmitting piece 260 is provided with a third connecting hole 261, and the first connecting hole 216, the second connecting hole 224, and the third connecting hole 261 are configured to receive the same fastener for threaded connection. This arrangement enables a fixed connection among the three components while reducing the number of fastening operations and openings required.

In some embodiments, with reference to FIG. 4, the conductive massage piece 240 is provided with a second groove 242 on its inner side. The second groove 242 is arranged around the rotational axis 241 of the conductive massage piece 240, and a portion of the conductive ball 230 is positioned within the second groove 242. This configuration achieves multiple effects: (i) it provides axial support and restriction of the conductive ball 230 relative to the rotational axis 241 of the conductive massage piece 240; (ii) it facilitates stable electrical transmission between the conductive ball 230 and the conductive massage piece 240; and (iii) the limiting fit between the conductive ball 230 and the second groove 242 ensures that the conductive massage piece 240 rotates along the predetermined trajectory of the second groove 242 without deviation, thereby enhancing stability.

Specifically, the bottom of the second groove 242 is formed as an arc-shaped surface. The diameter of the sphere defining the arc-shaped groove bottom is 4 to 10 times the diameter of the conductive ball 230. Within this range, the axial restriction strength between the conductive ball 230 and the second groove 242 is weakened appropriately, improving the rotational flexibility of the conductive massage piece 240 and reducing rotational resistance. If the diameter of the sphere defining the arc-shaped groove bottom is less than 4 times the diameter of the conductive ball 230, the contact area between the groove bottom and the conductive ball 230 increases, thereby increasing the rotational resistance of the conductive massage piece 240. Conversely, if the sphere diameter exceeds 10 times the diameter of the conductive ball 230, the support and restriction provided by the second groove 242 to the conductive ball 230 relative to the rotational axis 241 is weakened, which results in unstable rotational trajectories of the conductive massage piece 240 and the generation of noise and vibration during rolling.

The radius of the conductive ball 230 is 3 to 8 times greater than the groove depth of the second groove 242, ensuring that the conductive ball 230 maintains appropriate clearance and freedom of movement during rolling, thereby promoting smooth and stable operation.

Specifically, the surface roughness of at least one of the groove walls of the conductive ball 230 and the second groove 242 is less than or equal to 1 micron, which effectively reduces friction between the conductive ball 230 and the second groove 242 and improves rolling performance.

In some embodiments, with reference to FIG. 4, a gap is provided between the conductive connector 220 and the conductive massage piece 240, such that no direct contact occurs between the two components. This configuration reduces friction and wear between the conductive connector 220 and the conductive massage piece 240, thereby lowering the rotational resistance of the conductive massage piece 240 and improving operational smoothness.

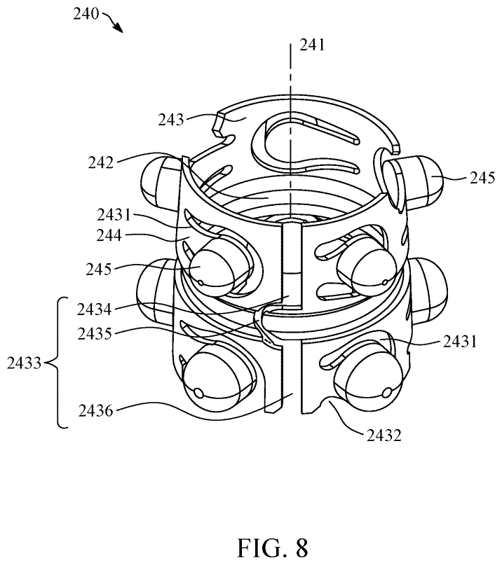

In some embodiments, with reference to FIGS. 3 and 8, the conductive massage piece 240 comprises a conductive sleeve 243, at least one elastic arm 244, and one or more massage protrusions 245. The conductive sleeve 243 is rotatably mounted on the insulating mounting piece 210 to provide radial constraint of the conductive massage piece 240. The conductive sleeve 243 is provided with a first through hole 2431, one end of the elastic arm 244 is connected to a wall of the first through hole 2431, and the other end of the elastic arm 244 is connected to the massage protrusion 245 at an interval from the conductive sleeve 243.

When the massage protrusion 245 is pressed against a user's body, the elastic arm 244 is deflected according to the shape and applied force of the contact surface, thereby allowing the massage protrusion 245 to undergo elastic expansion and contraction. This configuration ensures uniform distribution of massage force, reduces localized pressure, and improves massage comfort. The elastic arm 244 maintains a certain rigidity while enabling flexible deformation, thereby simulating multiple massage techniques (e.g., kneading, percussion, vibration) to enhance the massage effect.

In one embodiment, the conductive sleeve 243 is provided with at least one deformation gap 2433 extending axially, rendering the conductive sleeve 243 a non-closed ring structure. The non-closed ring structure allows greater elastic deformation of the conductive sleeve 243. During massage, when the massage protrusion 245 is pressed against the human body, the pressure is transmitted to the conductive sleeve 243, which undergoes elastic deformation to absorb and disperse the pressure, thereby reducing direct impact and discomfort. Simultaneously, the elastic deformation capability of the conductive sleeve 243 helps maintain reliable conductive contact with the conductive balls 230 during rotation and deformation, thereby optimizing the electrical conductivity of the massage mechanism 200.

As shown in FIG. 8, the deformation gap 2433 includes a first linear gap 2434, an arc-shaped gap 2435, and a second linear gap 2436. These gaps are sequentially connected and distributed along the rotation axis 241 of the conductive massage piece 240. The zigzag combination of linear and arc gaps allows stresses to be evenly distributed across the conductive sleeve 243 when subjected to external forces, thereby reducing stress concentration and improving structural strength and stiffness. Further, the non-linear shape of the deformation gap 2433 prevents axial misalignment of the conductive sleeve 243 relative to the rotation axis 241, ensuring proper positioning of the conductive ball 230. Additionally, the structure of the deformation gap 2433 includes concave-convex alignment on opposite sides of the arc-shaped gap 2435, which improves alignment and limits relative displacement, thereby reducing the risk of dislocation.

In one embodiment, two groups of massage protrusions 245 are provided along the rotation axis 241 and are distributed on opposite sides of the conductive ball 230. This arrangement balances forces during rotation of the conductive ball 230, reduces uneven wear of the conductive ball 230 and conductive massage piece 240, and provides more comprehensive massage coverage. Each group comprises one or more massage protrusions 245 distributed circumferentially around the rotation axis 241, thereby providing a greater number of massage points and improved precision.

In some embodiments, the massage protrusions 245 on the side distal from the handle 100 are larger than those on the proximal side. Since the distal side has a larger lever arm relative to the user's wrist (serving as the rotation support), larger massage protrusions 245 on the distal side deliver stronger massage forces for deep tissue relaxation, while smaller massage protrusions 245 near the handle 100 are suited for gentler massage in sensitive areas. The arrangement thereby adapts to varying massage needs of different body parts. Additionally, the larger circumferential space on the distal side allows for the use of larger massage protrusions 245 to perform wide-angle massage movements, whereas the narrower space near the handle 100 accommodates smaller massage protrusions 245.

In another embodiment, at least part of the first through hole 2431 includes a notch 2432 on the side opposite the conductive ball 230. The notch 2432 increases the deformation capacity of the corresponding wall of the first through hole 2431, thereby enlarging the elastic deformation range of the massage protrusion 245 disposed therein. For example, all first through holes 2431 on the upper side include notches 2432 to provide a larger elastic deformation space for stronger compression. Alternatively, in the lower side arrangement, two adjacent through holes 2431 are configured such that one has a notch 2432 to increase local deformation, while the other does not, thereby balancing structural strength with elastic performance.

In some embodiments, the conductive massage piece 240 is integrally formed such that the conductive sleeve 243, clastic arm 244, and massage protrusion 245 are manufactured as a single unit. This integrated molding reduces assembly operations, eliminates assembly errors, and improves positional accuracy and matching with the conductive ball 230. Moreover, the absence of joints reduces surface roughness and frictional resistance between the conductive sleeve 243 and conductive ball 230, resulting in smoother rotation.

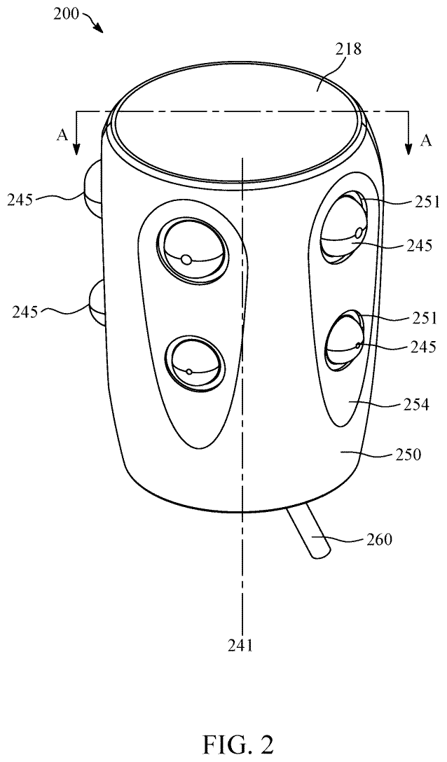

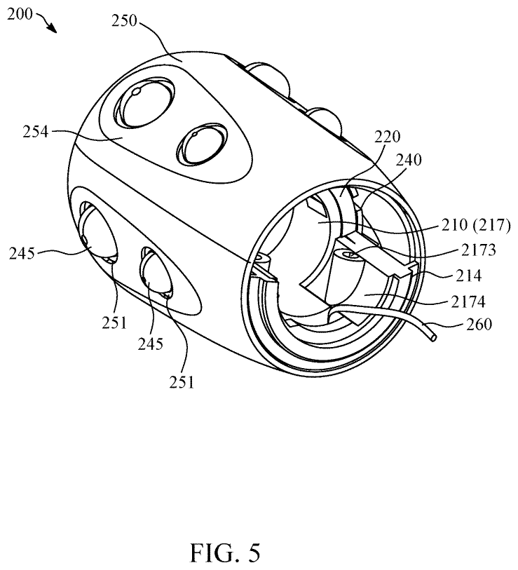

In certain embodiments, with reference to FIGS. 2, 3, and 5, the massage mechanism 200 further comprises an insulating housing 250 fixedly sleeved around the conductive sleeve 243. The insulating housing 250 is provided with a second through hole 251 through which the massage protrusion 245 protrudes outward. The insulating housing 250 electrically insulates the conductive sleeve 243 from the external environment, thereby preventing accidental contact with current and protecting the conductive sleeve 243 and internal components from moisture, dust, and contaminants. Additionally, the insulating housing 250 supports the conductive massage piece 240 without requiring additional connecting structures in direct contact with the insulating mounting piece 210, thus preserving the structural integrity and elastic performance of the conductive massage piece 240. The second through hole 251 also enables controlled positioning of the massage protrusion 245 for precise and targeted massage operations.

In one embodiment, both ends of the insulating housing 250 are axially limited by the insulating mounting piece 210 to ensure positional stability relative to the rotation axis 241. Specifically, at the end of the insulating housing 250 distal from the handle 100, a second limiting portion 252 is provided. The second limiting portion 252 abuts the insulating mounting piece 210, thereby restricting axial movement of the insulating housing 250 away from the handle 100 and providing swivel support between the insulating housing 250 and the conductive massage piece 240. The surface roughness of the second limiting portion 252 is less than or equal to 1 μm, thereby reducing frictional resistance between the insulating housing 250 and the insulating mounting piece 210.

At the opposite end of the insulating housing 250, i.e., the end closer to the handle 100, a third limiting portion 253 is provided. The third limiting portion 253 abuts the insulating mounting piece 210 to restrict movement of the insulating housing 250 toward the handle 100, thereby realizing one-way axial limitation and simultaneously providing rotational support between the insulating housing 250 and the conductive massage piece 240. The third limiting portion 253 also has a surface roughness of ≤1 μm to minimize friction. In certain embodiments, the insulating mounting piece 210 is further provided, at its end near the handle 100, with a first flange 214, and the third limiting portion 253 is rotatably supported on the first flange 214.

In another embodiment, the insulating housing 250 is further provided with an avoidance plane 254, and the second through hole 251 is disposed on the avoidance plane 254. The avoidance plane 254 allows the massage protrusion 245 to extend beyond the plane surface, thereby enhancing its ability to contact the human body, providing greater clastic deformation space, and enabling massage operations of varying amplitudes.

In some embodiments, with reference to FIGS. 3, 5, and 7, the insulating mounting piece 210 comprises a middle cage 217 and an end cap 218. One end of the middle cage 217 is fixedly connected to the handle 100, while the opposite end is detachably connected to the end cap 218. Such a detachable connection facilitates assembly and maintenance by allowing installation of the insulating housing 250 or the conductive massage piece 240, after which the end cap 218 is secured to provide axial limitation.

Specifically, the end cap 218 is provided with a hook 2181, while the middle cage 217 is provided, at the end distal from the handle 100, with a card slot 2171. The hook 2181 is detachably engaged in the card slot 2171, enabling tool-free assembly and disassembly. Additionally, the end cap 218 is provided with a guide block 2182, and the middle cage 217 is correspondingly provided with a guide groove 2172. The guide block 2182 engages the guide groove 2172 to achieve precise positioning during assembly. The hooks 2181 and guide blocks 2182 are circumferentially alternately distributed around the end cap 218 to further improve assembly reliability.

In some embodiments, with reference to FIG. 7, the interior of the insulating mounting piece 210 is provided with a reinforcing rib 219. The reinforcing rib 219 enhances the structural strength of the insulating mounting piece 210, thereby providing stable support for both the conductive massage piece 240 and the insulating housing 250. This configuration ensures that, even when the insulating mounting piece 210 is subjected to stress during massage operation, deformation is effectively prevented, thereby maintaining operational stability and durability.

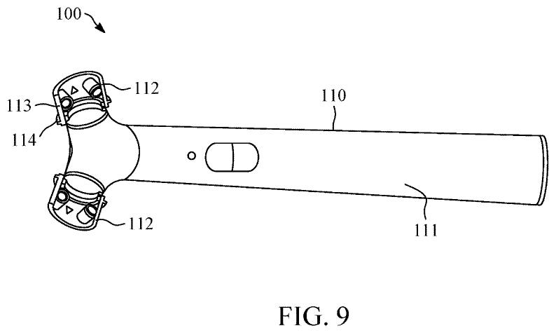

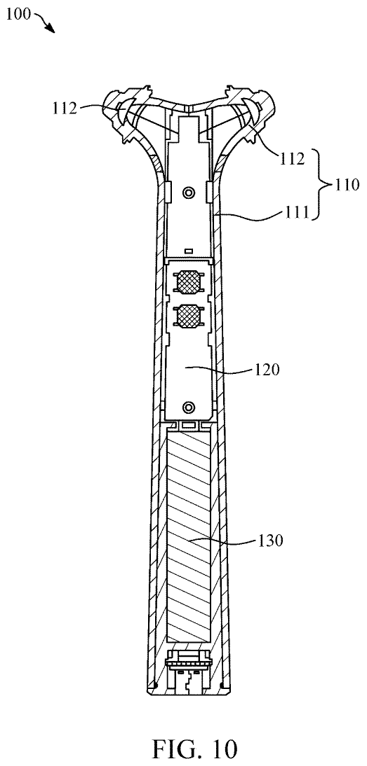

In some embodiments, with reference to FIGS. 1, 9, and 10, the handheld massage device comprising a handle 100 including a gripping portion 111 and two connecting branches 112 extending from the gripping portion 111 arranged at an angle with respect to one another. The two connecting branches 112 are symmetrically arranged relative to a central axis of the gripping portion 111. Each connecting branch 112 is provided with a corresponding massage mechanism 200, thereby forming a dual massage structure. The first massage mechanism is disposed on a first connecting branch 112 and electrically connected to a positive terminal of a power source 130, and the second massage mechanism is disposed on a second connecting branch 112 and electrically connected to a negative terminal of the power source 130. When both massage mechanisms 200 contact the human body, a microcurrent circuit is formed between them, thereby realizing microcurrent massage functionality.

In one embodiment, the handle 100 comprises a handle shell 110, within which are mounted a circuit board 120 and the power source 130. The circuit board 120 is electrically connected to the power source 130 and to the conductive connector 220. The power source 130 supplies electrical power to the circuit board 120, which then distributes power to the conductive connector 220 and subsequently to the conductive massage piece 240, thereby enabling microcurrent massage functionality.

The power source 130 provides a portable power source that allows the conductive massage piece 240 to be energized at any time and in any location, thereby expanding the range of use of the handheld massage device and improving user convenience.

In one embodiment, the connecting branch 112 is provided with a fourth connecting hole 113, and the insulating mounting piece 210 is provided with a fifth connecting hole 2173 corresponding to the fourth connecting hole 113. A fastener is detachably threaded through the fourth connecting hole 113 and the fifth connecting hole 2173, thereby enabling quick and detachable assembly of the massage mechanism 200 onto the handle 100.

Specifically, the connecting branch 112 has a half-shell shape, and the end of the insulating mounting piece 210 is provided with a connecting tab 2174. The connecting tab 2174 is bonded to the connecting branch 112 so as to form a cylindrical connection, thereby increasing the overall contact area and improving structural stability.

The connecting branch 112 is further provided with a second flange 114. The second flange 114, together with the connecting tab 2174 and the first flange 214 of the insulating mounting piece 210, forms an annular flange structure, which is configured to provide rotational support for the insulating housing 250.

A stimulation element can be embedded within or adjacent to the conductive massage piece 240 or in the connecting branches 112 to provide additional therapeutic effects. When in use, the user holds the gripping portion 111 of the handle 100 and positions the massage mechanisms against the skin, whereby the device simultaneously delivers mechanical massage through rotation, electrotherapy via electrical conduction, and enhanced stimulation through the embedded element. This integration allows the device to provide multi-modal treatment, including relaxation, muscle stimulation, improved circulation, and skin rejuvenation, thereby expanding its functional versatility for healthcare, cosmetic, and personal wellness applications.

In one embodiment of the invention, the stimulation element is selected from the group consisting of a phototherapy component, magnotherapy component, micro-current component, heating element, cooling element, Peltier element, vibrational element for massage, ultrasonic wave component, or other forms of therapeutic components.

Heating and cooling elements in the device operate by converting electrical energy into controlled temperature effects that act directly on the skin. The heating element, typically made of a resistive material, generates heat when an electric current passes through it. This heat is transferred to the massage piece or ball in contact with the skin. In contrast, the cooling element employs a peltier element, which creates a temperature differential when current flows through its semiconductor junctions. The cold side of the Peltier module is connected to the massage piece, producing a cooling effect. By integrating both heating and cooling functions, the device enables versatile thermal therapy that complements electrotherapy and massage, enhancing comfort and therapeutic outcomes for the user.

A user operates the device by first holding the handle 100, which houses the power source 130, and positioning the conductive massage piece 240 against the desired area of skin. When the device is activated, electrical current flows from the power source 130 through the conductive connector 220 and the conductive ball 230 to the conductive massage piece 240. By gently moving the conductive massage piece 240 over the skin, the user receives both the mechanical benefits of massage and the therapeutic effects of electrotherapy, either individually or in combination, depending on the selected mode of use.

The device has industrial applicability in cosmetic and skincare treatments to improve blood circulation, enhance skin tone, and aid anti-aging therapies.

The device has industrial applicability in the healthcare, wellness, and personal care sectors, where it is used for physiotherapy and rehabilitation to relieve pain, stimulate muscles, and support recovery.

It is also applicable in the sports and fitness industry for muscle relaxation and faster recovery after exercise.

It is also applicable in the home healthcare market as a convenient, portable solution for massage and electrotherapy without professional supervision.

Furthermore, its design is suitable for integration into consumer-grade and clinical-grade medical devices, making it valuable for manufacturers in the therapeutic and wellness device industry.

The foregoing embodiments are presented as preferred examples of the invention and are not intended to limit the scope of the present disclosure. Any modifications, equivalent substitutions, or improvements made within the spirit and principles of the invention shall fall within the scope of protection of the present claims.

1. A handheld massage device, comprising:
a handle including a gripping portion and at least two connecting branches extending from the gripping portion, arranged at an angle with respect to one another;
a first massage mechanism disposed on a first connecting branch and electrically connected to a positive terminal of a power supply; and
a second massage mechanism disposed on a second connecting branch and electrically connected to a negative terminal of the power supply,
wherein the first massage mechanism and the second massage mechanism are configured to contact a user's skin simultaneously to provide at least one physical massage or electrotherapy.

2. The handheld massage device of claim 1, wherein the at least two connecting branches are symmetrically arranged relative to a central axis of the gripping portion.

3. A handheld massage device, comprising:
a handle including a gripping portion and at least two connecting branches extending from the gripping portion, each of the at least two connecting branches is mounted with a massage mechanism;
wherein each massage mechanism comprises:
an insulating mounting piece;
a conductive massage piece rotatably supported by the insulating mounting piece and configured to contact a user's skin;
a conductive connector electrically connected to the conductive massage piece, configured to receive power from a power source accommodated within the handle; and
a conductive ball disposed between the conductive connector and the conductive massage piece, the conductive ball being in rolling contact with both the conductive connector and the conductive massage piece to facilitate smooth rotation and stable electrical conduction.

4. The handheld massage device of claim 3, further comprising an insulating housing disposed between the conductive massage piece and the insulating mounting piece.

5. The handheld massage device of claim 3, wherein the conductive massage piece is rotatably fitted onto the insulating mounting piece by a sleeve and a groove arrangement, the groove accommodating the conductive ball for smooth rotation.

6. The handheld massage device of claim 4, wherein the conductive massage piece comprises a conductive sleeve, at least one elastic arm, and one or more massage protrusions, wherein the at least one elastic arm connects the conductive sleeve to the one or more massage protrusions to provide flexible deformation.

7. The handheld massage device of claim 6, wherein the conductive sleeve has at least one axial deformation gap forming a non-closed ring to allow elastic deformation.

8. The handheld massage device of claim 6, the insulating housing fixedly sleeved around the conductive sleeve, the housing having a through hole through which the one or more massage protrusion extends.

9. The handheld massage device of claim 3, wherein the insulating mounting piece comprises at least one groove and a stop portion configured to limit axial or circumferential movement of the conductive massage piece.

10. The handheld massage device of claim 9, wherein the groove and the stop portion maintain stable rotational alignment of the conductive massage piece and conductive ball.

11. The handheld massage device of claim 3, wherein the conductive connector is sleeved onto the insulating mounting piece and electrically connected to the power source.

12. The handheld massage device of claim 3, wherein the conductive connector is in rolling contact with the conductive ball and the conductive massage piece throughout rotation.

13. The handheld massage device of claim 3, wherein a plurality of conductive balls is disposed circumferentially between the conductive connector and the conductive massage piece to provide multi-point electrical conduction and rotational stability.

14. The handheld massage device of claim 3, wherein the conductive connector and insulating mounting piece comprise cooperating limiting grooves or connecting holes to secure the conductive connector relative to the insulating mounting piece.

15. The handheld massage device of claim 3, wherein the conductive massage piece comprises a second groove on an inner side, the conductive ball being partially confined within the second groove.

16. The handheld massage device of claim 3, wherein the conductive massage piece rotates relative to the handle while the conductive ball maintains rolling contact to reduce friction and wear.

17. The handheld massage device of claim 3, further comprising one or more stimulation elements in each of the massage mechanisms to provide one or more therapies to the user's skin.

18. The handheld massage device of claim 17, wherein the stimulation element is selected from a group consisting of, but is not limited to, a phototherapy, micro-current, magneto therapy, cooling, heating, vibration, electrical pulses, or other forms of therapeutic output.

19. A method of massaging and applying electrotherapy to a user's skin, the method comprising:
providing a handheld massage device having a handle with a power source, at least one conductive massage piece, and an insulating mounting piece supporting the conductive massage piece;
transmitting electrical current from the power source through a conductive connector and a conductive ball to the conductive massage piece; and
contacting the conductive massage piece with the user's skin to provide at least one of the massage or electrotherapy.

20. The method of claim 19, wherein the conductive ball is housed in a groove of the insulating mounting piece to provide positional stability.
